(19) United States
(12) Patent Application Publication
(10) Pub. No.: US 2010/0074845 A1
(43) Pub. Date: Mar. 25, 2010

(54) ENHANCED SENSITIVITY CARBON NANOTUBES AS TARGETED PHOTOACOUSTIC MOLECULAR IMAGING AGENTS

(76) Inventors: Sanjiv S. Gambhir, Portola Valley, CA (US); Hongjie Dai, Cupertino, CA (US); Zhuang Liu, Stanford, CA (US); Adam de la Zerda, Stanford, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/552,313

(22) Filed: Sep. 2, 2009

Related U.S. Application Data
(60) Provisional application No. 61/093,555, filed on Sep. 2, 2008.

(51) Int. Cl. A61K 49/00; C07D 209/56; C07K 5/08; C07K 16/46; C07H 21/00; C07H 1/00; C07J 1/00; C07D 239/00; C07D 473/00; C07C 53/00

(52) U.S. Cl. 424/9.1; 548/427; 530/331; 530/391.3; 536/22.1; 536/123.1; 552/502; 554/1; 544/242; 544/264

(57) ABSTRACT

The present disclosure provides contrast photoacoustic probes, and compositions comprising such probes, designed to non-invasively detect and monitor various disease states, or targets within a subject human or animal. The probes are designed to be optically excited in tissue, ultimately generating thermal energy, which is transformed into acoustic energy by the response of the aqueous environment in the subject to the thermal emissions. The acoustic energy (sound) can then be detected by suitably applied transducers and digitally transformed into images indicating the location of the probe in the subject. One aspect of the disclosure encompasses photoacoustic probes that comprise: a carbon nanotube and a plurality of dye molecules bound to the carbon nanotube. The probes may further comprise a targeting moiety for localizing the probe at the site of a specific target. Another aspect of the present disclosure encompasses methods of detecting a target in animal or human subject, comprising: delivering a photoacoustic probe to a subject, allowing the photoacoustic probe to selectively bind to a target of the subject; and illuminating the system with an optical energy absorbable by the photoacoustic probe to generate an acoustic signal; and detecting the acoustic signal, thereby detecting the target in the subject.

### CROSS-REFERENCE TO RELATED APPLICATIONS

This application claims priority to U.S. Provisional Patent Application Serial No. 61/093,555 entitled “PHOTOACOUSTIC PROBES AND METHODS OF IMAGING” filed on Sep. 2, 2008, the entirety of which is hereby incorporated by reference.

### STATEMENT REGARDING FEDERALLY SPONSORED RESEARCH OR DEVELOPMENT

This invention was made with Government support under Contract/Grant Nos.: NCI CCNE U54, NCI ICMIC P50 CA 114747, and CA119367, awarded by the NCI (National Cancer Institute) of the United States Government. The Government has certain rights in this invention.

### TECHNICAL FIELD

The present disclosure is generally related to enhanced photoacoustic probes, methods of synthesis thereof, and methods of use in imaging targeted tissues.

### BACKGROUND

Photoacoustic techniques are investigative methods in which excitation laser pulses are absorbed in a target absorber to produce an acoustic response. The acoustic waves generated act as carriers of information relating to the light absorption properties of the target absorber and can be used to describe its constituents and structure. Applications include the characterization and imaging of biological tissue and non-destructive testing of materials and structures. While photoacoustic techniques offer an inherently powerful means of characterizing a target, their practical implementation can be problematic using conventional acoustic methods, particularly due to poor contrasting within the generated image.

Photoacoustic imaging as an emerging imaging modality overcomes, to a great extent, the resolution and depth limitations of optical imaging while maintaining the high-contrast of optics (Xu & Wang (2006) Rev. Sci. Instrum. 11: 041101-043100). When a short light pulse is used to illuminate tissues, the light is scattered and absorbed as it propagates through the tissues. The absorbed light is converted into heat, which in return causes the material to locally expand, creating a pressure wave. The pressure wave can then be detected by an ultrasound system placed outside the subject of interest.

By measuring the pressure waves from several positions, a full tomographic image can be reconstructed. This way, light only has to propagate into the tissue, and sound, which is minimally absorbed and scattered by tissues in low frequencies, propagates out of the tissue. Therefore, the depth of imaging can reach to about 5 cm, which is a significant increase compared to optical imaging techniques (Ku & Wang (2005) Opt. Lett. 30: 507-509). Photoacoustic imaging of living subjects has been used to image endogenous signals such as melanomas (Oh et al., (2006) J. Biomed. Opt. 11: 34032), thermal burns (Zhang et al., (2006) J. Biomed. Opt. 11: 054033), and oxygenation levels of blood (Wang et al., (2006) J. Biomed. Opt. 11: 024015).

However, most diseases will not manifest an endogenous photoacoustic contrast. Therefore, to fully utilize the potential of photoacoustic imaging, it is necessary to inject an exogenous photoacoustic contrast agent (a molecular imaging agent) that targets the diseased area(s) in the subject of interest. The ideal molecular imaging agent will have a sufficiently large optical absorption cross section to maximize the agent's photoacoustic signal, but yet be small enough to escape uptake by the reticuloendothelial system (RES), specifically the liver and the spleen. However, designing such an imaging agent is not trivial since a particle's absorption cross section and its size are highly correlated.

Recently, it has been shown that single walled carbon nanotubes (SWNTs) have utility as photoacoustic contrast agents (De Ia Zerda et al., (2008) Nat. Nanotechnol. 3: 557-62). SWNTs have strong light absorption characteristics and may act as photoacoustic contrast agents. SWNTs can be made as small as 1 nm in diameter but yet their length can extend to hundreds of nanometers increasing their absorption cross section and their intrinsic photoacoustic contrast. This unique geometry of SWNTs led to several applications of SWNTS in nanomedicine including drug delivery and photothermal therapy.

### SUMMARY

The utility of an in vivo contrast agent depends on preferential accumulation of the agent in target tissue and achievement of sufficient signal-to-noise ratios to yield satisfactory image resolution. The present disclosure provides novel contrast probes, and compositions comprising such probes, designed to non-invasively detect and monitor various disease states, or targets within a subject human or animal. The probes herein described are designed to be optically excited in tissue, ultimately generating thermal energy, which is transformed into acoustic energy by the response of the aqueous environment in the subject to the thermal emissions. The acoustic energy (sound) can then be detected by suitably applied transducers and digitally transformed into images indicating the location of the probe in the subject.

One aspect of the present disclosure, therefore, encompasses photoacoustic probes that comprise: a carbon nanotube and a plurality of dye molecules bound to said carbon nanotube, where the probe has the characteristic of being able to absorb optical energy and to convert the absorbed optical energy to thermal energy.

Another aspect of the present disclosure encompasses methods of detecting a target in a subject, comprising: delivering a photoacoustic probe to a subject, wherein the photoacoustic probe comprises a carbon nanotube, a plurality of dye molecules, and a targeting moiety, wherein the plurality of dye molecules and the targeting moiety are bound to said carbon nanotube, and wherein the probe has the characteristic of being able to absorb optical energy and being able to convert the absorbed optical energy to thermal energy to produce an acoustic signal; allowing the photoacoustic probe to selectively bind to a target of the subject; illuminating the system with an optical energy absorbable by the photoacoustic probe, thereby generating an acoustic signal; and detecting the acoustic signal, thereby detecting the target in the subject.

Still another aspect of the disclosure encompass kits comprising a photoacoustic probe according to the disclosure, packaging, and instructions for the use of the photoacoustic probe for the enhanced photoacoustic imaging of a region of a subject human or animal.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates a photoacoustic imaging instrument. A tunable pulsed laser (Nd:YAG laser and OPO) illuminated the subject through a fiber optic ring light. The photoacoustic signals produced by the sample were acquired using a 5 MHz focused transducer. A precision xyz-stage was used to move the transducer and the fiber ring along a planar 2D trajectory. The time of arrival and the intensity of the laser pulses were recorded using a silicon photodiode. This information could then be used to synchronize the acquisition and compensate for pulse-to-pulse variations in laser intensity. The analog photoacoustic signals were then amplified using a preamplifier and digitized using an oscilloscope.

FIG. 2 is a graph illustrating the optical absorbance spectra of SWNTs. The optical absorbance spectra of plain SWNTs (solid) and SWNT-RGD (dashed) were measured from 500-900 nm. The spectra suggest that the RGD peptide conjugation does not perturb the optical properties of the SWNT.

FIG. 3 is a graph illustrating the results of SWNT cell uptake studies. U87MG cells incubated with SWNT-RGD showed 75% higher SWNT signalling than did control U87MG cells incubated with plain SWNT, and 195% higher SWNT signal than HT-29 cells that were incubated with SWNT-RGD. “*” indicates p<0.05. U87MG cells incubated with saline only showed significantly lower signal than other groups (“**” indicates p<0.05 compared to other groups on the graph).

FIG. 4 illustrates a comparison between photoacoustic imaging using SWNTs and fluorescence imaging using QDs. A cylindrical inclusion filled with a mixture of SWNTs and QDs at equal concentrations was positioned 4.5 mm below the surface of a tissue-mimicking phantom. The digital photographic image (middle) of a horizontal slice through the phantom illustrates that the inclusion is 4.2 mm across. Fluorescence (top right) and photoacoustic (bottom right) digital images of the phantom are also shown. The dotted circle in the fluorescence digital image illustrates the true location of the inclusion. The photoacoustically generated digital image (right, bottom) represents a horizontal slice in the 3D image, 5 mm below the phantom surface. The estimated diameter of the inclusion in the fluorescence image was 11.5 mm (full-width half max), whereas the photoacoustic image accurately estimated the inclusion to be 4.2 mm across.

FIG. 5 shows a graph illustrating the optical absorption spectrum of single-walled carbon nanotubes (SWNT) (bottom line), SWNT conjugated to ICG molecules (SWNT-ICG) (top line), and SWNT conjugated to QSY-21 molecules (SWNT-QSY) (middle line).

FIG. 6 is a graph illustrating the optical absorption spectra of plain SWNT, SWNT-ICG-RGD, and SWNT-ICG-RAD probes. The spectral overlap between SWNT-ICG-RGD and SWNT-ICG-RAD suggests that the peptide conjugation does not perturb their spectra. Optical Absorption Spectrum is equivalent to Photoacoustic Signal strength.

FIG. 7 is a graph illustrating the results of SWNT-ICG cell uptake studies. U87MG cells incubated with SWNT-ICG-RGD showed over 95% higher signal than U87MG cell incubated with SWNT-ICG-RAD in the first 4 time points, and then dropped to 35% for 3 and 4 hours incubation times (p<0.05 for each time point independently).

FIG. 8 shows a series of digital images illustrating photoacoustic signals following intravenous injecting of tumor-bearing mice with SWNT-ICG-RGD and SWNT-QSY21-RGD. A significantly higher photoacoustic signal is detected at 4 hr post-injection, compared to the pre-injection control.

The drawings are described in greater detail in the description and examples below.

Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, biochemistry, biology, molecular biology, recombinant DNA techniques, pharmacology, imaging, and the like, which are within the skill of the art. Such techniques are explained fully in the literature. In particular, See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells and Enzymes” (IRL Press, (1986)); B. Perbal, “A Practical Guide To Molecular Cloning” (1984), each of which is incorporated herein by reference.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

The terms “administration” and “administering” as used herein refer to introducing an probe embodiment of the present disclosure to a subject. The preferred route of administration of an embodiment of the present disclosure is intravenously. However, any route of administration, such as oral, topical, subcutaneous, peritoneal, intra-arterial, inhalation, vaginal, rectal, nasal, introduction into the cerebrospinal fluid, or instillation into body compartments can be used.

The term “subject” as used herein refers to humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, particularly pets such as dogs and cats. For diagnostic or research applications, a wide variety of mammals will be suitable subjects, including rodents (e.g., mice, rats, hamsters), rabbits, primates, and swine such as inbred pigs and the like.

The term “detectable” as used herein refers to a change in or an occurrence of, a signal that is directly or indirectly detectable either by observation or by instrumentation and the presence or magnitude of which is a function of the presence of a target in the test sample. The term “detectable” refers to the ability to the capacity of a signal to be detected over the background signal. Although, typically, a detectable response is an optical response resulting in a change in the wavelength distribution patterns or intensity of absorbance or fluorescence or a change in light scatter, fluorescence quantum yield, fluorescence lifetime, fluorescence polarization, a shift in excitation or emission wavelength or a combination of the above parameters, the probes of the present disclosure are detectable by their emission of acoustic energy imparted to a surrounding aqueous medium, i.e. the tissues, cells and fluids of a subject human or animal. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “detecting” refers to detecting a signal generated by one or more photoacoustic probes. It should be noted that reference to detecting a signal from a photoacoustic probe also includes detecting a signal from a plurality of photoacoustic probes. In some embodiments, a signal may only be detected that is produced by a plurality of photoacoustic probes. Additional details regarding detecting signals (e.g., acoustic signals) are described below.

The term “dye compound” as used herein refers to s a fluorescent molecule, i.e., one that emits electromagnetic radiation, especially of visible light, when stimulated by the absorption of incident radiation. The term includes, but is not limited to, fluorescein, a xanthene dye having an absorption maximum at 495 nanometers. A related fluorophore is Oregon Green, a fluorinated derivative of fluorescein. The term further includes bora-diaza-indecene, rhodamines, and cyanine dyes.

A “rhodamine” is a class of dyes based on the rhodamine ring structure. Rhodamines include (among others): TETRAMETHYLRHODAMINE™, and carboxy tetramethyl-rhodamine (TAMRA). Rhodamines are established as natural supplements to fluorescein based fluorophores, which offer longer wavelength emission maxima and thus open opportunities for multicolor labeling or staining. The term is further meant to include “sulfonated rhodamine,” a series of fluorophores known as ALEXA FLUOR™ dyes (Molecular Probes, Inc). These sulfonated rhodamine derivatives exhibit higher quantum yields for more intense fluorescence emission than spectrally similar probes, and have enhanced photostability, absorption spectra matched to common laser lines, pH insensitivity, and a high degree of water solubility.

“Cyanines” are a family of cyanine dyes, Cy2, Cy3, Cy5, Cy7, and their derivatives, based on the partially saturated indole nitrogen heterocyclic nucleus with two aromatic units being connected via a polyalkene bridge of varying carbon number. These probes exhibit fluorescence excitation and emission profiles that are similar to many of the traditional dyes, such as fluorescein and tetramethylrhodamine, but with enhanced water solubility, photostability, and higher quantum yields. The excitation wavelengths of the Cy series of synthetic dyes are tuned specifically for use with common laser and arc-discharge sources, and the fluorescence emission can be detected with traditional filter combinations. Cyanine dyes are available as reactive dyes or fluorophores coupled to a wide variety of secondary antibodies, dextrin, streptavidin, and egg-white avidin. The cyanine dyes generally have broader absorption spectral regions than members of the Alexa Fluor family.

A “quencher” is a compound that can modulate the emission of a fluorophore. A quencher may itself be a fluorescent molecule which emits fluorescence at a characteristic wavelength. Thus a fluorophore may act as a quencher when appropriately coupled to another dye and vice versa. In this case, increase in fluorescence from the acceptor molecule, which is of a different wavelength to that of the donor label, will also indicate binding of the ABP. Alternatively, the acceptor does not fluoresce (dark acceptor). True quenchers such as dabcyl (“D”), the “Black Hole Quenchers” (“BHQs”), and the QSY family of dyes (QSY-5, QSY-7, or QSY-9) are broad spectrum absorbing molecules that appear dark or even black in color, because they absorb many wavelengths of light and do not re-emit photons.

Such acceptors include (4(4′dimethylaminophenylazo)benzoic acid (DABCYL), methyl red, and QSY-7™. The structure of QSY 7™, a non-fluorescent diarylrhodamine derivative, is illustrated in Kumaraswamy et al., US Patent Publication 2005/0014160, which is incorporated herein by reference in its entirety. Typical fluorophore/quencher compounds include certain rhodamine dyes or Cy5.

Diazo dyes of the BHQ series, which are referred to as “Black Hole Quenchers” (International Patent Publication No. WO 01/86001), provide a broad range of absorption, which overlaps, well with the emission of many fluorophores. The QSY series dyes from Molecular Probes, Inc are another series of dark quenchers used extensively as quenching reagents (see for example U.S. Pat. No. 6,399,392).

The term “acoustic signal” refers to a sound wave produced by one of several processes, methods, interactions, or the like (including light absorption) that provides a signal that can then be detected and quantitated with regard to its frequency and/or amplitude. The acoustic signal can be generated from one or more photoacoustic probes. In an embodiment, the acoustic signal may need to be the sum of each of the individual photoacoustic probes or groups of photoacoustic probes. In an embodiment, the acoustic signal can be generated from a summation, an integration, or other mathematical process, formula, or algorithm, where the acoustic signal is from one or more photoacoustic probes. In an embodiment, the summation, the integration, or other mathematical process, formula, or algorithm can be used to generate the acoustic signal so that the acoustic signal can be distinguished from background noise and the like.

The term “acoustic detectable signal” is a signal derived from a probe of the present disclosure that absorbs light and converts absorbed energy into thermal energy, thereby generating an acoustic signal through a process of thermal expansion. The acoustic detectable signal is detectable and distinguishable from other background acoustic signals that are generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background.

The term “illuminating” as used herein refers to the application of any light source, including near-infrared (NIR), visible light, including laser light capable of exciting dyes and nanotubes of the embodiments of the photoacoustic probes herein disclosed.

The term “in vivo imaging” as used herein refers to methods or processes in which the structural, functional, or physiological state of a living being is examinable without the need for a life ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “kit” as used refers to a packaged set of related components, typically one or more compounds or compositions, and typically includes containers for the components of the kit, instructions for their use according to the methods of the present disclosure, advertising, trademarks, etc.

The term “biocompatible” as used herein in conjunction with the terms monomer or polymer, refers to polymers and probes that do not substantially interact with the tissues, fluids and other components of the body in an adverse fashion in the particular application of interest.

The term “optical energy” as used herein refers to electromagnetic radiation between the wavelengths of about 350 nm to about 800 nm and which can be absorbed by the dyes or carbon nanotubes of the embodiments of the photoacoustic probes of the disclosure. The term “optical energy” may be construed to include laser light energy or non-laser energy.

The term “thermal energy” as used herein refers to electromagnetic radiation of wavelengths between about 700 nm and about 1000 nm and which can increase the temperature of a medium exposed to such radiation.

The term “aqueous medium” as used herein refers to any composition or medium comprising water in the free or liquid state, that is, not bound in a dry medium such as water of crystallization. In the context of the systems receiving the photoacoustic probes of the present disclosure, an aqueous medium can be, but is not limited to, a biological cell, a biological tissue or organ, or a biological fluid, including such as blood, interstitial fluid surrounding a tissue in an animal or human body, and the like.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

### Discussion

The present disclosure provides novel contrast probes, and compositions comprising such probes, designed to non-invasively detect and monitor various disease states, or targets within a subject human or animal. The probes herein described are designed to be optically excited in tissue, ultimately generating thermal energy, which is transformed into acoustic energy by the response of the aqueous environment in the subject to the thermal emissions. The acoustic energy (sound) can then be detected by suitably applied transducers and digitally transformed into images indicating the location of the probe in the subject. The utility of an in vivo contrast agent depends on preferential accumulation of the agent in target tissue and achievement of sufficient signal-to-noise ratios to yield satisfactory image resolution.

Photoacoustic imaging of living subjects offers significantly higher spatial resolution at increased tissue depths compared to purely optical imaging techniques. Intravenously injected single walled carbon nanotubes (SWNTs) of the present disclosure having a dye incorporated into the structure thereof can be used as targeted photoacoustic imaging agents in living mice using, for example, the RGD peptide moiety to target αᵥβ₃ integrins.

The present disclosure encompasses photoacoustic imaging agents based on SWNT, but further comprising a plurality of dye molecules incorporated into or onto the SWNTs. The inclusion of the dye molecules into the probe greatly enhances the input of optical energy to the carbon nanotube, resulting in enhancement of the output thermal energy by the nanotube. The probes of the present disclosure may also be conjugated to targeting moieties that can preferentially localize the probe to a desired target, such as a cell or tissue. The result is a detectable, target localized, acoustic signal with a significantly enhanced signal-background noise ratio, and a concomitant increase in the contrast quality of the acoustical image generated. For example, one embodiment of the probes of the disclosure comprises a photoacoustic contrast agent based on SWNTs that have indocyanine green (ICG) molecules bound to their surface (SWNT-ICG). This increases the photoacoustic contrast by up to 20 times compared to plain SWNTs due to much stronger light absorption characteristics. Furthermore, the absorption peak of the SWNT-ICG particles is located at 780 nm, a wavelength at which tissue optical absorption and therefore photoacoustic background signal are minimal.

While the embodiments described herein are focused on the use of single wall carbon nanotubes, it is further contemplated that the probes may comprise multi-walled carbon nanotubes. Although the following primarily refers to SWNTs, embodiments of the present disclosure include SWNTs and MWNTs. Reference to SWNTs in many parts of the disclosure is done for clarity, and is not limiting to only SWNTs and it can include MWNTs.

In all embodiments of the present disclosure, it is contemplated that other dyes may be useful besides ICG and QSY. The choice of the most appropriate dye may be determined according to the properties of the nanotube probe, the means of administration to the subject, and the like. Furthermore, the targeting moiety may be any moiety able to selectively bind to a desired target within the subject, including, but not limited to, an antibody, a peptide, an oligonucleotide, a protein such as a cytokine, and the like. The targeting moiety may be attached to the carbon nanotubes directly via covalent bonds that do not significantly reduce or eliminate the target binding capacity of the moiety, or via a linker or tether molecule.

The present disclosure further provides methods of imaging in a subject by administering to the subject a pharmaceutically acceptable composition comprising any of the photoacoustic probes herein disclosed. After sufficient time has been allowed to elapse for the administered probe to contact and be concentrated by a targeted cell or tissue in the subject, the human or animal may be irradiated with a light, such as a laser light, at a wavelength absorbed by the dye of the photoacoustic probe. The generated acoustic signal may then be detected by a suitably configured transducer for conversion of the acoustic signal into a visual image.

Accordingly, embodiments of the present disclosure include photoacoustic probes, methods of making photoacoustic probes, methods of imaging, and the like. Embodiments of the photoacoustic probes are able to detect one or more targets (e.g., cells, tissue, tumors, chemicals, enzymes, and the like) by detecting the generation of an acoustic signal. Embodiments of the photoacoustic probe include a carbon nanotube (single walled carbon nanotube (SWNT) or multi-walled nanotube (MWNT)) having a plurality of dyes bound to the carbon nanotube.

For example, and not intended to be a limiting embodiment of the photoacoustic probes according to the present disclosure, either QSY21 or Indocyanine Green, with absorption peaks at about 707 nm and about 780 nm, respectively, can be attached to a carbon SWNT, whereupon the photoacoustic signal of these imaging agents can be enhanced by about 70 times, as compared with plain SWNTs. SWNTs can also, for example, be coupled to RGD-comprising peptides through a linker such as polyethylene glycol-5000 grafted to a phospholipid, as described for example in De la Zerda et al., (2008) Nature Nanotech. 3: 557-562, which is incorporated herein by reference in its entirety. The dye molecules QSY21 or ICG molecules, however, appeared to be bound to the surface of each SWNT non-covalently through pi-pi stacking interactions.

In vitro serum stability of such particles can be measured. Cell uptake and blocking studies with U87MG cells have verified that nanoparticles bearing the RGD peptide moiety can bind selectively to αᵥβ₃ integrin. SWNT-QSY21 and SWNT-ICG that were injected subcutaneously to living mice (n=4) can be visualized at concentrations as low as 3 nM, representing at least about a 70-fold enhancement in sensitivity over what could be achieved with plain SWNTs. Finally, it can be shown that upon intravenous administration, RGD-targeted SWNT-QSY21 and SWNT-ICG selectively bind to integrin αᵥβ₃-expressing U87MG tumor-bearing mice, unlike non-targeted SWNT-QSY21 and SWNT-ICG probes.

Embodiments of the photoacoustic probe can include a single walled carbon nanotube (SWNT) having a plurality of dyes bound to the SWNT. Relative to SWNT not including the dyes, embodiments of the present disclosure have increased absorption at the appropriate peaks (SWNT absorbs at a different wavelength than embodiments of the present disclosure) by a factor of 5 or more, 10 or more, 15 or more, 20 or more, 30 or more, 40 or more, 50 or more, 60 or more, or 70 or more. In addition, embodiments of the present disclosure increase the photoacoustic signal by a factor of 5 or more, 10 or more, 15 or more, 17 or more, or 20 or more, relative to SWNT not including the dyes. Thus, embodiments of the present disclosure are advantageous over plain SWNTs.

It should also be noted that a targeting moiety can be attached (e.g., directly or indirectly) to the photoacoustic probe so an image of a specific target can be correlated with the acoustic signal. In other words, an image of the target can be created using the acoustic signal from photoacoustic probe concentrated to a specific target.

Embodiments of the photoacoustic probes can be used to provide high acoustic contrast for imaging. In this regard, the photoacoustic probes can be used for imaging anatomical and/or physiological events in a host. Embodiments of the present disclosure enable the imaging of anatomical and/or physiological and/or molecular events in vitro or in vivo using photoacoustic techniques and methods. The image acquired using the photoacoustic probes can be used to illustrate the concentration and/or location of the photoacoustic probes. In embodiments where the photoacoustic probes is labeled with a targeting moiety that has an affinity for a target (e.g., tumor), the image acquired can be correlated with the location and/or dimensions of the target.

The photoacoustic probes of the present disclosure may include a single walled carbon nanotube (SWNT) having a plurality of dye molecules bound to the SWNT. In an embodiment of the present disclosure, the photoacoustic probe may include a multi-walled carbon nanotube (MWNT) having a plurality of dye molecules bound to the MWNT. Embodiments of the photoacoustic probe has the characteristic of being able to absorb optical energy and being able to convert the absorbed energy to thermal energy to produce an acoustic signal.

In particular, the SWNT and the dye compounds combine to absorb the optical energy and convert it to thermal energy to produce a detectable acoustic signal when the probe is in a suitable environment, and in particular an aqueous environment. Typically, the dye molecules are non-covalently bound to the SWNT. Although not intending to be bound by theory, the dye molecules are bound to the SWNT non-covalently through pi-pi stacking interactions. An advantage of the pi-pi stacking is the ultra-high loading (e.g., 1 gram of SWNT can load 5-10 grams of ICG molecules) that can be attained. It is contemplated, however, that the dye molecules can be bound to the SWNT via covalent conjugation, whereupon only to about 300 or so dye molecules can be attached to each SWNT.

In another embodiment of the present disclosure, the photoacoustic probe includes a single walled carbon nanotube (SWNT) having a plurality of dye molecules bound to the SWNT and a targeting moiety attached (e.g., directly or indirectly) to the SWNT. The photoacoustic probe has the characteristic of being able to absorb optical energy and being able to convert the absorbed energy to thermal energy to produce an acoustic signal. In particular, the SWNT and the dye compounds combine to absorb the optical energy and convert it to thermal energy to produce a detectable acoustic signal. In addition, the targeting moiety can be used to direct the photoacoustic probe to a target. Detection of the acoustic signal can be correlated with an image of the target (e.g., a tumor). In an embodiment, the image of the target can be used to determine the location and/or dimensions of the target.

### SWNTs

Carbon nanotubes (CNTs) suitable for use in the photoacoustic probes of the present disclosure are allotropes of carbon with a cylindrical nanostructure. Nanotubes have been constructed with length-to-diameter ratio of up to 28,000,000:1, which is significantly larger than any other material. Nanotubes are members of the fullerene structural family, which also includes the spherical buckyballs. The ends of a nanotube might be capped with a hemisphere of the buckyball structure. Their name is derived from their size, since the diameter of a nanotube is on the order of a few nanometers, while they can be up to several millimeters in length. Nanotubes are categorized as single-walled nanotubes (SWNTs) and multi-walled nanotubes (MWNTs).

The nature of the bonding of a nanotube is described by applied quantum chemistry, specifically, orbital hybridization. The chemical bonding of nanotubes is composed entirely of sp² bonds, similar to those of graphite. This bonding structure, which is stronger than the sp³ bonds found in diamonds, provides the molecules with their unique strength. Nanotubes naturally align themselves into “ropes” held together by Van der Waals forces.

Most single-walled nanotubes (SWNT) have a diameter of close to 1 nanometer, with a tube length that can be many millions of times longer. The structure of a SWNT can be imagined by wrapping a one-atom-thick layer of graphite called graphene into a seamless cylinder. The way the graphene sheet is wrapped is represented by a pair of indices (n,m) called the chiral vector. The integers n and m denote the number of unit vectors along two directions in the honeycomb crystal lattice of graphene. If m=0, the nanotubes are called “zigzag”. If n=m, the nanotubes are called “armchair”. Otherwise, they are called “chiral”.

Single-walled nanotubes are an important variety of carbon nanotube because they exhibit electric properties that are not shared by the multi-walled carbon nanotube (MWNT) variants. SWNTs can be excellent electrical conductors. SWNTs can be excited to produce tangential vibration upon exposure to optical energy.

Multi-walled nanotubes (MWNT) have multiple rolled layers (concentric tubes) of graphite. There are two models which can be used to describe the structures of multi-walled nanotubes. In the ‘Russian Doll’ model, sheets of graphite are arranged in concentric cylinders, e.g. a (0.8) single-walled nanotube (SWNT) within a larger (0.10) single-walled nanotube. In the ‘Parchment’ model, a single sheet of graphite is rolled in around itself, resembling a scroll of parchment or a rolled newspaper. The interlayer distance in multi-walled nanotubes is close to the distance between graphene layers in graphite, approximately 3.3 Å.

The morphology and properties of double-walled carbon nanotubes (DWNT) are similar to SWNT, but their resistance to chemicals is significantly improved. This is especially important when functionalization is required (grafting of chemical functions at the surface of the nanotubes) to add new properties to the CNT. In the case of SWNT, covalent functionalization can break some C═C double bonds, leaving “holes” in the structure on the nanotube and thus modifying both its mechanical and electrical properties. In the case of DWNT, only the outer wall is modified. (See, Yakobson & Smalley, American Scientist, Vol. 85, July-August, 1997, pp. 324-337, which is incorporated herein by reference).

In an embodiment, the carbon nanotubes including SWNTs and MWNTs may have diameters of about 0.6 nanometers (nm) up to about 3 nm, about 5 nm, about 10 nm, about 30 nm, about 60 nm or about 100 nm. In an embodiment, the single-wall carbon nanotubes may have a length of about 50 nm up to about 1 millimeter (mm), or greater. In an embodiment, the diameter of the single-wall carbon nanotube is about 2 to 5 nm and has a length of about 50 to 500 nm. Embodiments of the MWNT can include 2 or more concentric walls, 5 or more concentric walls, 10 or more concentric walls, 20 or more concentric walls, or 40 or concentric more walls, or at least one ‘parchment’ rolled wall.

### Dyes

Dye compounds suitable for use in the photoacoustic probes of the present disclosure such as small molecule dyes can include, but are not limited to, fluorescent dyes or non-fluorescent quenchers such as, but not limited to, dabcyl, non-fluorescent pocilloporins, diarylrhodamine derivatives (e.g., QSY-7, QSY-9, and QSY-21), polyaromatic-azo quenchers (e.g., QSY-35, BHQ-1, BHQ-2 and BHQ-3), indocyanine dyes (e.g., indocyanine green dyes and derivatives thereof), and bisazulene derivatives, an isothiocyanate dye, a multi-sulfur organic dye, a multi-heterosulfur organic dye, a benzotriazole dye, a thiacyanine dye, a dithiacyanine dye, a thiacarbocyanine dye, a dithiacarbocyanine dye, malachite green isothiocyanate, tetramethylrhodamine-5-isothiocyante, X-rhodamine-5-isothiocyanate, X-rhodamine-6-isothiocyanate, 3,3′-diethylthiadicarbocyanine iodide, and the like.

In embodiments of the photoacoustic probes of the disclosure, the SWNT can be attached to one type of dye compound. In other embodiments, the SWNT can be attached to two or more types of dye compound, whereupon the probes may respond to more than one wavelength of irradiating optical energy.

The amount of dye compound bound to an SWNT can be, but is not limited to, about 1 to 10000 dye compounds per SWNT, about 100 to 10000 dye compounds per SWNT, about 500 to 5000 dye compounds per SWNT, and about 1000 to 4000 dye compounds per SWNT. Incorporation of the dye molecules into the lattice structure of SWNTs and MWNTs is described in Example 2, below.

### Targeting Moiety

In general, the targeting moiety can include, but is not limited to, polypeptides (e.g., proteins such as, but not limited to, antibodies (monoclonal or polyclonal, and the selectively binding fragments Fab, Fab′, F(ab′)₂, single chain Fv (S_{c}Fv) and Fv fragments thereof)), nucleic acids (both monomeric and oligomeric), polysaccharides, sugars, fatty acids, steroids, purines, pyrimidines, ligands, or combinations thereof. The targeting moiety selected for incorporation into the probes of the present disclosure can have an affinity for one or more targets. In general, the desired target can include, but is not limited to, a cell type, a cell surface, extracellular space, intracellular space, a tissue type, a tissue surface, the vascular, a polypeptide, a nucleic acid, a polysaccharide, a sugar, a fatty acid, a steroid, a purine, a pyrimidine, a hapten, a ligand, and the like, related to a condition, disease, or related biological event or other chemical, biochemical, and/or biological event of the sample or host.

The targeting moiety can be selected based on the target selected and the environment the target is in and/or conditions that the target is subject to. The targeting moiety can be specific or non-specific. The specific-targeting moiety can be selected to have an affinity (e.g., an attraction to) for a target such as, but not limited to, a specific protein, a cell type, a receptor, a transporter, an antigen, and a saccharide (e.g., a monosaccharide, a disaccharide and a polysaccharide), as well as other molecules that can interact with the targeting moiety. The specific targeting moiety can include, but is not limited to, an antibody, an antigen, a polypeptide, an aptamer, a small molecule, and ligands, as well as other molecules that bind to the target.

For example, the targeting moiety is a RGD containing peptide (e.g., a peptide that includes RGD but may include one or more (e.g., two) amino acids). The RGD containing peptide can include 1 or more (e.g., 2, 3, 4, 5, 6, 7, 8, or more) RGD peptide units. The RGD peptide unit can be a cyclic peptide containing the Arg-Gly-Asp amino acid sequence. The term “cyclic peptide” refers to a head-to-tail cyclized peptide and/or a cyclized peptide via one or more disulfide bonds. As mentioned above, the RGD containing peptide can include one or more amino acids on either side of the RGD region. In an embodiment, the RGD containing peptides include 2 additional amino acids, 3 additional amino acids, 5 additional amino acids, 10 additional amino acids, 25 additional amino acids, 50 additional amino acids, 100 additional amino acids, or 500 additional amino acids.

The non-specific targeting moiety can be selected to do one or more of the following: enter a cell or a cell type, enter the vasculature, enter extracellular space, enter intracellular space, have an affinity for a cell surface, diffuse through a cell membrane, react with a non-specified moiety on the cell membrane, enter tumors due to leaky vasculature, and the like. In an embodiment, the non-specific targeting moiety can include a chemical, biochemical, or biological entity that facilitates the uptake of the photoacoustic probe into a cell. The non-specific targeting moiety can include, but is not limited to, cell penetrating peptides, polyamino acid chains, small molecules, and peptide mimics.

The targeting moiety can be linked, directly or indirectly, to the SWNT in a manner described above using a stable physical, biological, biochemical, and/or chemical association. In general, the targeting moiety can be independently linked via chemical bonding (e.g., covalently or ionically), biological interaction, biochemical interaction, and/or otherwise associated with the SWNT in a manner described above. The targeting moiety can be independently linked using a link such as, but not limited to, a covalent link, a non-covalent link, an ionic link, a chelated link, as well as being linked through interactions such as, but not limited to, hydrophobic interactions, hydrophilic interactions, charge-charge interactions, π-stacking interactions, combinations thereof, and like interactions.

In addition, the agent can also include, but is not limited to, a drug, a therapeutic agent, radiological agent, photosensitizers, a small molecule drug, and combinations thereof, that can be used to treat the target molecule and/or the associated disease and condition of interest. The drug, therapeutic agent, and radiological agent can be selected based on the intended treatment as well as the condition and/or disease to be treated. In an embodiment, the photoacoustic probe can include two or more agents used to treat a condition and/or disease. In addition, the detection of the photoacoustic probe can be used to ensure the delivery of the agent or drug to its intended destination as well as the quantity of agent or drug delivered to the destination.

In particular, the photoacoustic probes can be used in in-vivo diagnostic and/or therapeutic applications such as, but not limited to, targeting diseases and/or conditions and/or imaging diseases and/or conditions. For example, one or more embodiments of the photoacoustic probes can be used to identify the type of disease or condition, identify the presence of one or more compounds associated with the disease or condition, locate the proximal locations of the disease or condition, and/or deliver agents (e.g., drugs) to the diseased cells (e.g., cancer cells, tumors, and the like) in living animals.

### Linker

The targeting moiety incorporated into the photoacoustic probes of the present disclosure can be attached to the SWNT via a linker such as, but not limited to, a polyethylene glycol polymer, a dextran, a peptide, or the like. In some embodiments, the linker can be a polyethylene glycol polymer. In these embodiments, the targeting moiety may be attached to the SWNT using polyethylene glycol-5000 grafted phospholipids (PL-PEG), where the hydrophobic lipid chains stably bind to the nanotube surface, while the hydrophilic PEG can extend towards a surrounding aqueous phase environment to impart water solubility and biocompatibility to the nanotubes. Other types of surfactants or amphiphilic polymers can also be used to functionalize SWNTs, For example, but not intended to be limiting, PEGylated polypyrene can be used. In another embodiment, PEGylated fatty acid with lipid chain length greater than about 20 can be used. Other covalent reactions can be used to attach the PEG to SWNT and functionalization chemistry can be used to accomplish this end. It should also be noted that non-covalent functionalization can be used to link the PEG to the SWNT. The use of PEG-based linkers, and methods of their attachment to an SWNT has been described in De la Zerda et al., (2008) Nature Nanotech. 3: 557-562, incorporated herein by reference in its entirety.

The PEG can be a linear PEG, a multi-arm PEG, a branched PEG, or any combination thereof. The molecular weight of the PEG can be about 1 kDa to 100 kDa, about 1 kDa to 50 kDa, about 1 kDa to 40 kDa, about 1 kDa to 30 kDa, about 1 kDa to 20 kDa, about 1 kDa to 12 kDa, about 1 kDa to 10 kDa, and about 1 kDa to 8 kDa. When used in reference to PEG moieties, the word “about” indicates an approximate average molecular weight and reflects the fact that there will normally be a certain molecular weight distribution in a given polymer preparation.

The amount of PEG polymer bound to a SWNT can be, but is not limited to, about 20 to 500 PEG polymer compounds per SWNT. For example, in some embodiments of the probes, the amount of PEG polymer bound to a SWNT can be about 50 to 250 PEG polymer compounds per SWNT. In other embodiments, the amount of PEG polymer bound to a SWNT can be about 100 to 200 PEG polymer compounds per SWNT.

### Acoustic Detection System

The acoustic energy can be detected and quantified in real time using an appropriate detection system. The acoustic signal can be produced by one or more photoacoustic probes.

One possible system is described in the following references: J. Biomedical Optics 11: 024015; Optics Letters, 30: 507-509, which are included herein by reference. The acoustic energy detection system can include, but is not limited to, for example, a 5 MHz focused transducer (25.5 mm focal length, 4 MHz bandwidth, F number of 2.0, depth of focus of 6.5 mm, lateral resolution of 600 μm, and axial resolution of 380 μm. A309S-SU-F-24.5-MM-PTF, Panametrics) that can be used to acquire both pulse-echo and photoacoustic images. In addition, high resolution ultrasound images can also be simultaneously acquired using a 25 MHz focused transducer (27 mm focal length, 12 MHz bandwidth, F number of 4.2, depth of focus of 7.5 mm, lateral resolution of 250 μm, and axial resolution of 124 μm. V324-SU-25.5-MM, Panametrics). Other detection strategies including capacitive micromachined ultrasonic transducers (CMUT) arrays can also be used to detect the acoustic signal.

### Methods of Use

The present disclosure further relates generally to methods for studying (e.g., detecting, localizing, and/or quantifying) cellular events, molecular events, in vivo cell trafficking, stem cell studies, vascular imaging, tumor imaging, biomolecule array systems, biosensing, biolabeling, gene expression studies, protein studies, medical diagnostics, diagnostic libraries, microfluidic systems, and delivery vehicles. The present disclosure also relates to methods for multiplex imaging of multiple events substantially simultaneously inside a subject (e.g., a host living cell, tissue, or organ, or a host living organism) using one or more photoacoustic probes.

In short, a photoacoustic probe according to the present disclosure may be included in a pharmaceutically acceptable composition suitable for delivery to a subject human or animal. Such compositions may further include a pharmaceutically acceptable carrier well known to those in the art. Such photoacoustic probes having a targeting moiety can be introduced to the system (sample or host) using known techniques (e.g., injection, oral administration, and the like) to determine if the system includes one or more targets (e.g., a cell, a cell marker, a tissue, a tissue in a pathological state associated with a specific target marker, and the like).

After an appropriate lapse of time, during which unassociated photoacoustic probes can be sufficiently cleared from the appropriate area, region, or tissue of interest, the sample (e.g., living cell, tissue, or organ) or host may be illuminated with an optical energy. The detection of the acoustic signal can be measured using systems described herein. The production of the acoustic signal indicates that the target is present in the sample or host.

The photoacoustic probes disclosed herein can be used to study, image, diagnose the presence of, and/or treat cancerous cells, precancerous cells, cancer, or tumors. For example, the presence of the cancerous cells, precancerous cells, cancer, or tumors can provide insight into the appropriate diagnosis and/or treatment. It should be noted that photoacoustic probes could include agents specific for other diseases or conditions so that other diseases or conditions can be imaged, diagnosed, and/or treated using embodiments of the present disclosure. In an embodiment, other diseases and/or conditions can be studied, imaged, diagnosed, and/or treated in a manner consistent with the discussion below as it relates to cancerous cells, precancerous cells, cancer, and/or tumors.

In another embodiment, the photoacoustic probes include one or more agents to treat the cancerous cells, precancerous cells, cancer, or tumors. Upon measuring the acoustic signal, one can determine if the photoacoustic probe has coordinated with the cancerous cells, precancerous cells, cancer, or tumors. Embodiments of the photoacoustic probe can aid in visualizing the response of the cancerous cells, precancerous cells, cancer, or tumors to the agent.

In general, the photoacoustic probes can be used in a screening tool to select agents for imaging, diagnosing, and/or treating a disease or condition. In an embodiment, the photoacoustic probes can be used in a screening tool to select agents for imaging, diagnosing, and/or treating cancerous cells, precancerous cells, cancer, or tumors. The photoacoustic probes can be imaged and it can be determined if each agent can be used to image, diagnose, and/or treat cancerous cells, precancerous cells, cancer, or tumors.

### Kits

This disclosure encompasses kits that include, but are not limited to, photoacoustic probes packaging, and directions (written instructions for their use). The components listed above can be tailored to the particular disease, biological event, or the like, being studied, imaged, and/or treated (e.g., cancer, cancerous, or precancerous cells). The kit can further include appropriate buffers and reagents known in the art for administering various combinations of the components listed above to the host cell or host organism.

One aspect of the present disclosure, therefore, encompasses photoacoustic probes that comprise: a carbon nanotube and a plurality of dye molecules bound to said carbon nanotube, where the probe has the characteristic of being able to absorb optical energy and to convert the absorbed optical energy to emitted thermal energy.

In embodiments of the photoacoustic probe according to the disclosure, the emitted thermal energy has the characteristic of being able to generate an acoustic signal in an aqueous medium.

In embodiments of this aspect of the disclosure, the photoacoustic probes can further comprise a targeting moiety bound to the carbon nanotube.

In embodiments of this aspect of the disclosure, the carbon nanotube can be a single-walled nanotube (SWNT).

In other embodiments of this aspect of the disclosure, the carbon nanotube can be a multi-walled nanotube (MWNT).

In embodiments of this aspect of the disclosure, the carbon nanotube can have a diameter of about 0.6 nanometers (nm) to about 100 nm, and the carbon nanotube can have a length of about 50 nm to about 1 mm.

In other embodiments of this aspect of the disclosure, the carbon nanotube can have a diameter of about 2 nanometers (nm) to 5 nm, and the carbon nanotube can have a length of about 50 nm to about 500 nm.

In embodiments of the photoacoustic probes of this aspect of the disclosure, the dye compound can be selected from the group consisting of: a diarylrhodamine, a polyaromaticazo quencher, Blackberry Q, a bisazulene, an indocyanine, an indocyanine, a dabcyl, a non-fluorescent pocilloporins, an isothiocyanate dye, a multi-sulfur organic dye, a multi-heterosulfur organic dye, a benzotriazole dye, a thiacyanine dye, a dithiacyanine dye, a thiacarbocyanine dye, a dithiacarbocyanine dye, a malachite green isothiocyanate, a tetramethylrhodamine-5-isothiocyante, an X-rhodamine-5-isothiocyanate, an X-rhodamine-6-isothiocyanate, a 3,3′-diethylthiadicarbocyanine iodide, and a combination thereof.

In some embodiments of this aspect of the disclosure, the diarylrhodamine derivatives can be selected from the group consisting of: QSY-7, QSY-9, QSY-219.

In other embodiments of this aspect of the disclosure, the polyaromatic-azo quencher can be selected from the group consisting of: QSY-35, BHQ-1, BHQ-2 and BHQ-3.

In some embodiments of this aspect of the disclosure, the indocyanine dyes is an indocyanine green dye or a derivative thereof.

In one embodiment of the probes of the disclosure, the dye molecule is QSY21, and wherein the probe absorbs energy at about 707 nm.

In another embodiment of the probes of the disclosure, the dye molecule is Indocyanine Green, and wherein the probe absorbs energy at about 780 nm.

In the embodiments of the probes of the disclosure, the targeting moiety may comprise a peptide having the amino acid sequence arginine-glycine-aspartic acid (RGD).

In these embodiments of the probes of the disclosure, the targeting moiety can be selected from the group consisting of: a monoclonal antibody, a polyclonal antibody, an Fab fragment, an Fab′ fragment, an F(ab′)₂ fragment, a single chain Fv (ScFv) fragment, an Fv fragment, a nucleic acid, a polysaccharide, a sugar, a fatty acid, a steroid, a purine, a pyrimidine, and a small molecule ligand.

In the embodiments of the probes of the disclosure, the targeting moiety can be bound to the carbon nanotube via a linker.

In some embodiments, the targeting moiety can be bound to the carbon nanotube via a polyethylene glycol (PEG) polymer linker.

In embodiments of this aspect of the disclosure, the photoacoustic probe is in a probe composition, where the probe composition can further comprise a pharmaceutically acceptable carrier.

Another aspect of the present disclosure encompasses methods of detecting a target in animal or human subject, comprising: delivering a photoacoustic probe to a subject, wherein the photoacoustic probe comprises a carbon nanotube, a plurality of dye molecules, and a targeting moiety, wherein the plurality of dye molecules and the targeting moiety are bound to said carbon nanotube, and wherein the probe has the characteristic of being able to absorb optical energy and being able to convert the absorbed optical energy to emitted thermal energy to produce an acoustic signal in an aqueous medium; allowing the photoacoustic probe to selectively bind to a target of the subject; illuminating the system with an optical energy absorbable by the photoacoustic probe, thereby generating an acoustic signal; and detecting the acoustic signal, thereby detecting the target in the subject.

In embodiments of the methods this aspect of the disclosure, detection of the acoustic signal can be used to determine the presence and location of the target in the subject.

In embodiments of the methods this aspect of the disclosure, the methods can further comprise generating an image of the target by detecting the acoustic signal in the subject.

Still another aspect of the disclosure encompass kits comprising a photoacoustic probe according to the disclosure, packaging, and instructions for the use of the photoacoustic probe for the enhanced photoacoustic imaging of a region of a subject human or animal.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Photoacoustic Imaging Instrumentation

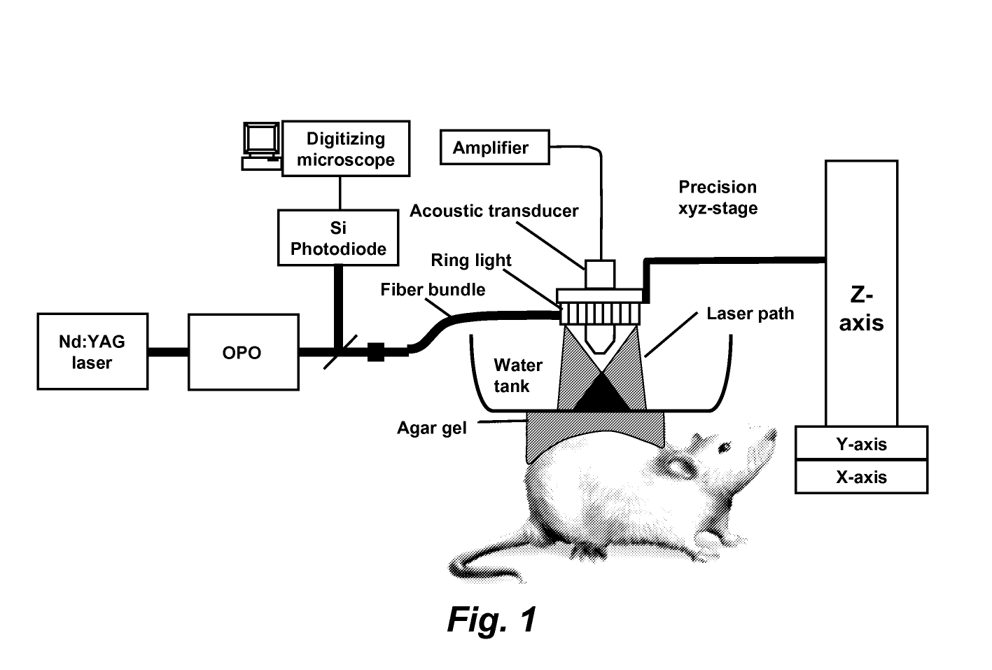

A photoacoustic system (as described by Vaithilingam et al., in (2007) Ultrasonics Symposium. IEEE 2413-2416, incorporated herein by reference in its entirety) and used in the detection and generation of the photoacoustic images according to the present disclosure, is illustrated in FIG. 1. In this system, a tunable pulsed laser with a repetition rate of 10 Hz and a pulse width of 5 ns (Nd:YAG Surelight-III-10 connected to Surelite OPO Plus, Continuum) illuminated the object through a fiber optic ring light (50-1353 Ringlight, Fiberoptic Systems Inc.). The average energy density of the laser at 690 nm wavelength was measured to be about 9 mJ/cm² at the target site, which was below the ANSI limitation for laser skin exposure (American National Standards Institute, (2000) ANSI Standard ZI36.1-2000, ANSI, Inc., New York).

A 5 MHz focused transducer (25.5 mm focal length, 4 MHz bandwidth, f2.0, depth of focus of 6.5 mm, lateral resolution of 600 μm, and axial resolution of 380 μm. A3095-SU-F-24.5-MM-PTF, Panametrics) was used to acquire both pulse-echo and photoacoustic images. In addition, high resolution ultrasound images were acquired using a 25 MHz focused transducer (27 mm focal length, 12 MHz bandwidth, f4.2, depth of focus of 7.5 mm, lateral resolution of 250 (μm, and axial resolution of 124 μm. V324-SU-25.5-MM, Panametrics).

A precision xyz-stage (U500, Aerotech Inc.) with a minimum step size of Iμm was used to move the transducer and the fiber ring along a planar 2D trajectory. At every position, the acquired signal was averaged over 16 laser pulses. The time of arrival and the intensity of the laser pulses were recorded using a silicon photodiode (DET10A, Thorlabs). This information was used to synchronize the acquisition and compensate for pulse-to-pulse variations in laser intensity. The analog photoacoustic signals were amplified using a tunable preamplifier (Panametrics) and digitized using an oscilloscope (Infmiium 54825A, Agilent).

The photoacoustic and ultrasound images were reconstructed as follows: the a-scan from each position of the transducer was band pass filtered with 100% fractional bandwidth, compensated for laser intensity variation and envelope detected. The a-scans were then combined to reconstruct a 3D intensity image of the target. No further post-processing was done on the images. The ultrasound images acquired using the 5 MHz and 25 MHz transducers were aligned together using small vertical translations so that the object's skin level matches in both images. Then, photoacoustic and the high frequency ultrasound images were analyzed, co-registered and displayed using AMIDE software (Loening & Gambhir (2003) Mol. Imaging. 131-137, incorporated herein by reference in its entirety).

### Mouse Arrangement in the Photoacoustic System.

Female nude mice were used for all the photoacoustic studies. The mice scanned in the photoacoustic system were fully anesthetized using isoflurane delivered through a nose-cone. As schematically shown in FIG. 1, prior to the photoacoustic scan, the areas of interest were covered with agar gel to stabilize the area and minimize any breathing and other motion artifacts. A saran-wrap water bath was placed on top of the agar gel. An ultrasonic transducer, placed in the water bath, was therefore acoustically coupled to the mouse tissues. This setup allowed the ultrasonic transducer to move freely in 3D while not applying any physical pressure on the mouse.

### Example 2

### SWNT-ICG-RGD Conjugate Synthesis

The synthesis of SWNT-RGD and SWNT-RAD is described in Liu et al., (2007) Nat. Nano. 2: 47-52, incorporated herein by reference in its entirety. SWNT-RGD and SWNT-RAD were then incubated with excess of ICG molecules dissolved in DMSO overnight. Unbound ICG molecules were removed from the solution by filtration of the SWNT particles. The SWNTs were 50-300 nm in length and 1-2 nm in diameter. The molar concentrations determined as described by Kam et al. (2005) Proc. Natl. Acad. Sci. USA. 102: 11600-11605, incorporated herein by reference in its entirety) are based on an average molecular weight of 170 kDa per SWNT (150 nm in length and 1.2 nm in diameter).

### Example 3

### Optical Characterization of SWNT-ICG

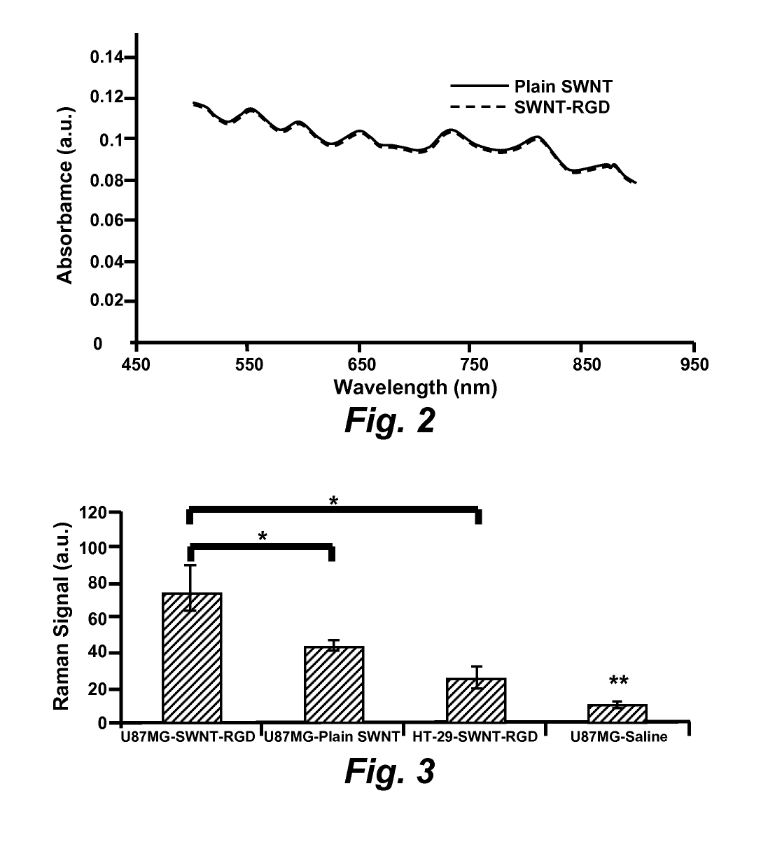

The optical absorption spectra of the SWNT and SWNT-RGD particles are shown in FIG. 2. The absorption spectra of both SWNT and SWNT-RAD were found to be almost identical, indicating that the presence of the RGD peptide moiety does not perturb SWNT optical properties.

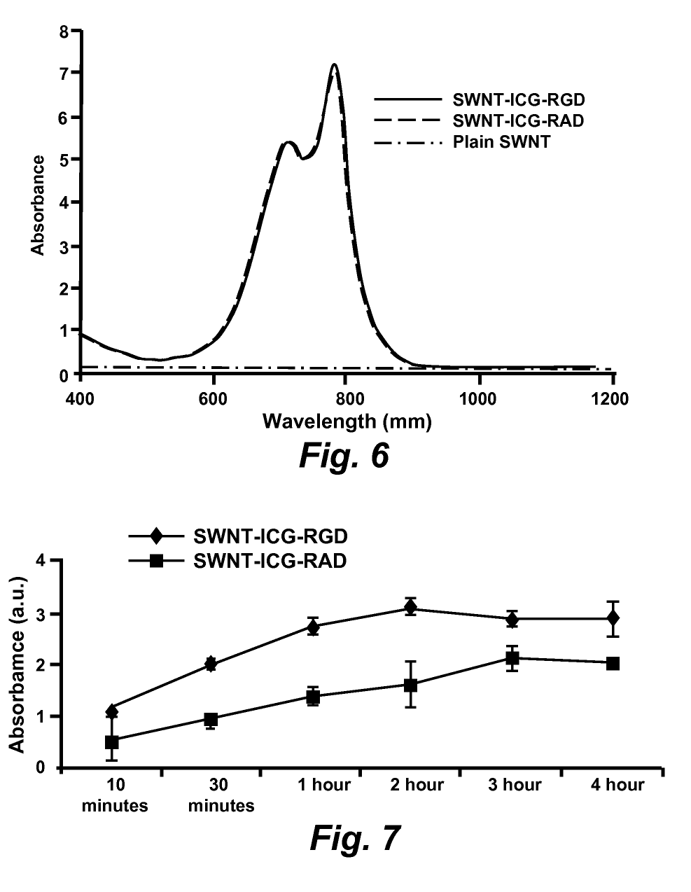

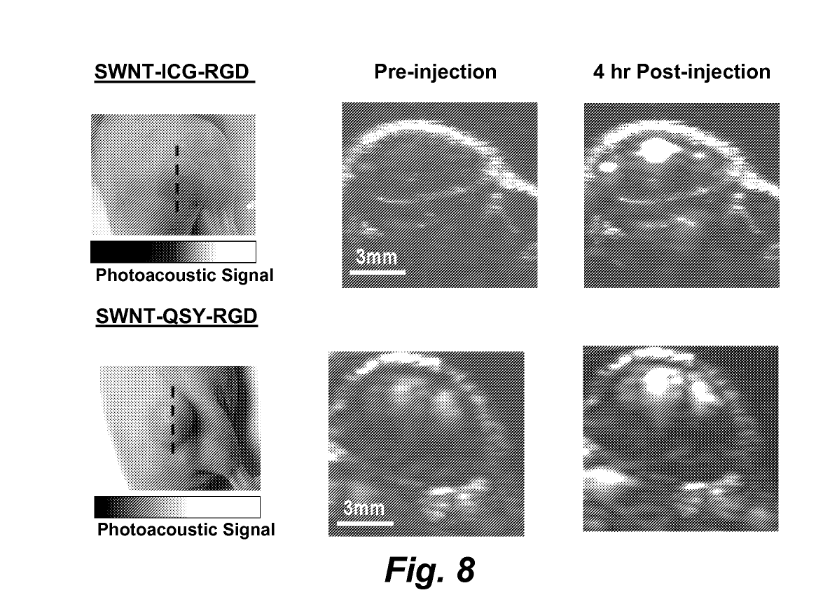

The optical absorption spectra of the SWNT-ICG particles is shown in FIG. 6. The absorption spectra of both SWNT-ICG-RGD and SWNT-ICG-RAD were found to be almost identical, indicating that the presence of the RGD or RAD peptide conjugated to the SWNT-ICG does not perturb the SWNT-ICG optical properties had no effect. The absorption spectra of the particles peak at 780 nm, and represent an almost 20-fold improvement in absorption over unmodified (plain) SWNTs. Since blood optical absorption is minimum at about 780 nm, the photoacoustic background signal is also a minimum, leading to even greater sensitivity than at other wavelengths.

### Example 4

### SWNT-ICG Serum Stability

2.5 nM of SWNT-ICG-RAD was incubated with 10% serum: 90% PBS. The optical absorbance of the solution at a wavelength of 780 nm was monitored every 3 minutes for 2.5 hrs. Control solutions included 10% serum only, or 2.5 nM of SWNT-ICG-RAD only. Throughout the 2.5 hour period, the absorbance of all solutions remained the same and did not deviate more than 5% (p<0.05).

### Example 5

### Cell Uptake of SWNT Probes

1.2×10⁶ αᵥβ₃ integrin-expressing U87MG cells were exposed to 100 μl of 600 nM SWNT-RGD. As a control, 1.2×10⁶ U87MG cells were exposed to the same volume and concentration of plain SWNT. Another 1.2×10⁶ U87MG control group was exposed to 1×PBS (1×PBS, pH 7.4, Invitrogen). Additionally, 1.2×10⁶ cells HT-29 cells, which do not express αᵥβ₃ integrin on their surface, were exposed to 100 μl of 600 nM SWNT-RGD (n=3 in all groups). The cells were exposed for 30 min, and then centrifuged. All excess liquid was removed and cells were washed with PBS twice. The cells were then suspended in 15 μl of liquid agar gel and scanned ex vivo using a Raman microscope.

SWNTs produce a very unique Raman signal, as described by Jorio et al., (2004) Philos. Transact. A. Math. Phy. Eng. Sci. 362: 2311-2336, incorporated herein by reference in its entirety, allowing a Raman microscope to detect low concentrations of SWNTs in cells. U87MG cells that were exposed to SWNT-RGD were found to have 75% higher signal than U87MG cells exposed to plain SWNT (p<0.05) and 195% higher signal than HT-29 cells exposed to SWNT-RGD (p<0.05). Cells exposed to saline only showed negligible signal compared to any of the groups (p<0.05), as shown in FIG. 3.

### Example 6

### Cell Uptake of SWNT-Fluorescent Dye-Containing Probes

Approximately 1×10⁶ αᵥβ₃-expressing cells (U87MG) were exposed to SWNT-ICG-RGD and SWNT-ICG-RAD for periods from about 10 min up to about 4 hours. Control cells were exposed to SWNT-ICG-RAD. After exposure, the cells were washed with saline to remove unbound particles and scanned for their absorbance at 780 nm using a highly sensitive spectrophotometer.

As shown in FIG. 7, after 2 hours of incubation, U87MG cells exposed to SWNT-ICG-RGD were found to have 95% higher absorbance than cells exposed to SWNT-ICG-RAD (p<0.05), indicating the specific binding of the RGD-targeted particles to the αᵥβ₃ receptor, in contrast with the control particles. Furthermore, after 2 hours of incubation, the number of particles bound to the cell reached a maximum, considered to be an optimal time point.

### Example 7

### Tissue Background Calculation

The SWNTs used had an absorbance A=6.2×10⁶ cm⁻M⁻¹ at 690 nm (measured using DU-640 spectrophotometer, Beckman Coulter). Assuming light absorption accounts for most of the absorbance of the SWNTs, then:

μ_{CA}(λ,C)=ln(10)×A(λ)×C

where μ_{CA} and C are the contrast agent optical absorption coefficient and concentration respectively.

Upon light exposure I to the absorber at wavelength λ, the absorber will produce a pressure wave P=Γ×I×μₐ(λ), where Γ is the Gruneisen coefficient and μₐ(λ) is the optical absorption coefficient of the absorber.

The optical absorption (and hence the background photoacoustic signal) of tissues varies between different locations in the body due to different amounts of oxyhemoglobin (HbO₂), hemoglobin (Hb), and melanin that leads to different optical absorption characteristics and, therefore, to different endogenous photoacoustic background signals. It was estimated that typical tissues with absorption coefficient of 0.1-1 cm⁻¹ will produce a background photoacoustic signal that is equivalent to the photoacoustic signal produced by 7-70 nM of SWNTs.

Importantly, in cases where background signal is mixed with the contrast agent signal (e.g., background cannot be measured prior to contrast agent administration or is not spectrally separated from the contrast agent signal), sensitivity criteria typically requires that the contrast agent signal will be greater than, or equal to, the tissue background signal. This requirement can be formulated as: P_{CA}≧P_{Tissue}, where P_{CA} and P_{Tissue} are the photoacoustic pressure wave from the contrast agent and the tissue respectively. Assuming the contrast agent does not affect the Gruneisen coefficient of the tissue, this criterion reduces to: μ_{CA}(λ)≧μ_{Tissue}(λ), where μ_{CA}(λ) and μ_{Tissue}(λ) are the optical absorption coefficients of the contrast agent and the tissue respectively.

### Calculation of Percentage Injected Dose Per Gram of Tissue

The photoacoustic signal produced by 50 nM of SWNTs was equivalent to the endogenous photoacoustic signal produced by tissues. Since mice injected with SWNT-RGD showed a 67% increase in photoacoustic signal produced by tumors, the SWNTs concentration in the tumor can be estimated to be about 33.5 nM. The mice were injected with 240 μmol of SWNT-RGD (200 μl at 1.2 μM concentration). Assuming that 1 mm³ of tissue weights 1 mg, the percentage injected dose per gram of tissue (% ID/g) is therefore about 14% ID/g.

### Example 8

### Characterization of SWNT Photoacoustic Properties

A gel phantom was prepared using 1% Ultrapure Agarose (Invitrogen) and 1% intra-lipid (Liposyn II 10%, Abbott Laboratories) to induce scattering into the phantom. After solution solidification, cylindrical wells 4.2 mm in diameter were created in the phantom. Plain (unmodified) SWNT was then mixed with warm liquid agar at a ratio of 1:4 (final concentration of the SWNG was 200 nM) and poured the solution into the wells. The same procedure was then repeated for SWNT-RGD.

After the agar solidified, the wells were covered by another thin layer of warm agar and allowed to solidify. A complete photoacoustic image of the phantom was acquired at wavelengths between 690-800 nm in 5 nm steps. The photoacoustic signals were compensated for laser power and photodiode response in the difference wavelengths, so that each measurement represented only the inherent photoacoustic signal produced by SWNTs. For image analysis, a 3D ROI was drawn over the SWNT in the phantom and the mean signal in the ROI was calculated. To test the linearity of the photoacoustic signal as a function of SWNT concentration, an agar-phantom was used with no scattering or absorbing additives (i.e. no intra-lipid).

SWNTs at increasing concentration were mixed with warm liquid agar in ratio of 1:3 to form SWNTs solutions at 25, 50, 100, 200, 300, 400 nM. Inclusions 3 mm under the phantom surface were filled with the various SWNTs solutions (three inclusions for each concentration, 100 μl per inclusion). A complete photoacoustic image of the phantom was acquired at 690 nm with step size of 0.5 mm. 3D cylindrical ROIs at the size of the inclusion were used to estimate the photoacoustic signal from each well.

### Comparison to Optical Fluorescence Imaging Using Quantum Dots

A gel phantom was prepared using 1% Ultrapure Agarose (Invitrogen) and 1% intra-lipid (Liposyn II 10%, Abbott Laboraties). After a 30 min wait for the agar-lipid solution to solidify, cylindrical wells were created in the phantom with a diameter of 4.2 mm. The wells were then filled with a mixture of QDs (Qdot(r) 800 ITK™ amino (PEG) quantum dots, Invitrogen), and plain SWNT at equal concentrations. Control wells were filled with QDs only or plain SWNT only. Liquid agar was then added to all wells at a ratio of 4:1 to allow the well content to solidify.

After the dilution with the liquid agar, the concentration of plain SWNT and QDs in the wells was 200 nM. After 30 min, a second layer, 4.5 mm in height, of warm agar-lipid liquid was poured. After a further 30 min, the phantom was scanned in a fluorescence imaging instrument Maestro (CRI). A band pass excitation filter centered around 645 nm and a 700 nm long pass emission filter were used for the scan. The tunable band pass filter was set to scan the fluorescence emission from the phantom at wavelengths between 700 nm to 950 nm. An exposure time of 300 ms was found to maximize the fluorescence signal from the QD-SWNT well while not saturating the camera. Maestro proprietary software was used to calculate the full-width half max (FWHM).

SNR was calculated as the maximal signal acquired from the well divided by the average signal in a small ROI drawn 14 mm away from the inclusion's center. Photoacoustic and ultrasound images of the phantom were then acquired. The laser wavelength was set to 690 nm and averaging of 16 laser pulses per photoacoustic a-scan was used. The lateral step size was set to 250 μm. The resulting photoacoustic image was analyzed using AMIDE software. The estimated depth of the inclusion was determined by overlaying the photoacoustic image on the ultrasound image which shows the surface of the agar-phantom. The estimated inclusion diameter was measured directly from the photoacoustic image and the image SNR was calculated as the photoacoustic signal at the inclusion area divided by the mean signal outside the inclusion.

### Example 9

### Comparison to Optical Fluorescence Imaging Using QDs

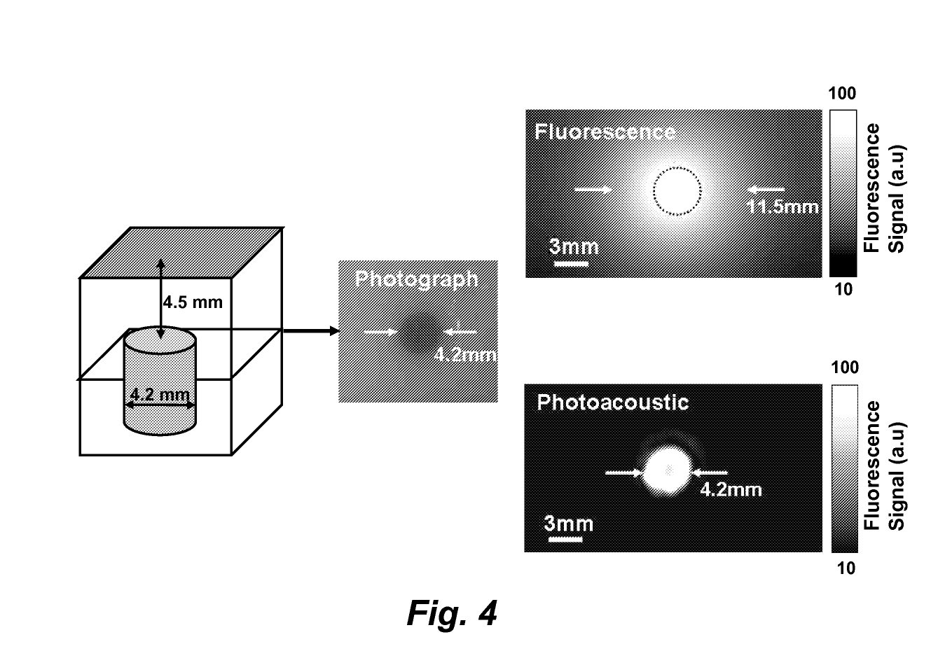

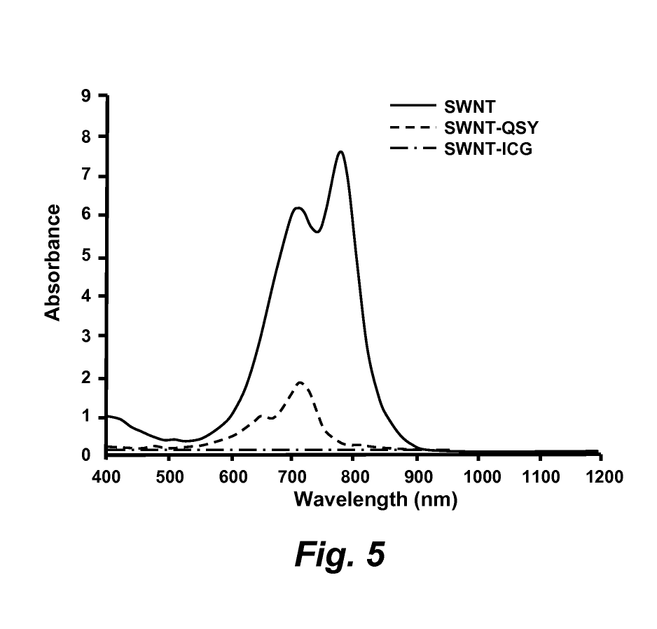

The photoacoustic strategy using the SWNT compositions of the present disclosure were compared to fluorescence imaging with quantum dots (QDs). The agar-based phantom was constructed with a scattering coefficient, μₛ⁻¹=1 μm⁻¹, similar to that of tissues and negligible absorption. The phantom had a cylindrical inclusion of 4.2 mm in diameter embedded 4.5 mm below the phantom surface, as shown in FIG. 4. The inclusion was filled with a mixture of SWNTs or QDs at 200 nM each. The QDs were approximately 30 nm in diameter with emission wavelength of 800 nm. The phantom was scanned under a fluorescence imaging instrument and under the photoacoustic imaging instrument as described in Example 1, FIG. 1, as shown in FIG. 4.

Control inclusions filled with plain SWNT only, or QDs only, showed no fluorescence signal and no detectable photoacoustic signal respectively. SWNTs are non-fluorescent at 800 nm (as discussed in Barone et al., (2005) Nat. Materials 4: 86-92, incorporated herein by reference in its entirety). Quantum dots, however, are highly fluorescent and therefore only minimal energy is available for heating and creating photoacoustic vibrations.

The fluorescence image (FIG. 4) showed a large blurred spot at the center of the phantom, with an estimated diameter of 11.5 mm (full width half max), whereas the photoacoustic imaging technique revealed the edges of the inclusion, and accurately estimated it's diameter to be 4.2 mm. Furthermore, the depth of the inclusion was accurately estimated in the photoacoustic image to be 4.5±0.1 mm. Depth estimation at this accuracy cannot be done using fluorescence imaging. Additionally, the signal to noise ratio (SNR), which is associated with sensitivity, was significantly higher in the photoacoustic image (SNR=38) than in the fluorescence image (SNR=5.3).

### Example 10

### Photoacoustic Detection of SWNTs in Living Mice

Plain SWNT at 6 different concentrations were mixed with matrigel (Matrigel Basement Membrane Matrix, Phenol Red-free, Becton Dickinson) at a 1:1 ratio creating plain SWNT solutions at 50, 100, 200, 300, 400 and 600 nM. The solutions were then injected subcutaneously (30 μl) to the lower back of mouse (n=3). After the matrigel solidified in its place (a few minutes) the back of the mouse was scanned under the photoacoustic system described in Example 1.

A photoacoustic image with lateral step size of 0.5 mm was acquired using the 5 MHz transducer at 690 nm wavelength. Following the photoacoustic scan, an ultrasound image was acquired using the 25 MHz transducer and the two images were then co-registered. Quantification of the photoacoustic signal was done by drawing a 3D ROI over the inclusion volume that is illustrated in the ultrasound image. The volume of the ROIs was kept at 30 mm³ (equivalent to the 30 μl that were injected).

### Example 11

Two groups of female nude mice (n=3 in each group), 6-8 weeks old were inoculated subcutaneously at their lower right back with 10⁷ U87MG cells (American Type Culture Collection, ATCC) suspended in 50 ml of saline (1×PBS, pH 7.4, Invitrogen). The tumors were allowed to grow to a volume of about 100 mm³. Before the injection of single-walled carbon nanotubes, photoacoustic and ultrasound images of the mice were taken. Photoacoustic excitation light was 690 nm. The single-walled carbon nanotubes were sonicated for 5 min under 1 W r.m.s. (Sonifier 150, Branson) to separate single-walled carbon nanotubes that may have aggregated. The mice were then injected with 200 ml of 1.2 μM single-walled carbon nanotubes into the tail-vein. During the injection the positioning of the mice was not changed. After injection, photoacoustic and ultrasound images were acquired at 0.5, 1, 2, 3 and 4 h post injection. The scanning area varied between mice depending on the tumor orientation, but typically was about 80 mm², with a step size of 0.25 mm. At 4 h post-injection, the mice were killed and their tumors surgically removed for further ex vivo analysis. The ultrasound images from the different time points were aligned with one another by vertically translating the images (translation was typically less than 0.5 mm). The same alignment was then applied to the photoacoustic images. Using AMIDE software, a 3D region of interest was drawn over the tumor volume (which was clearly illustrated in the ultrasound images). The mean photoacoustic signal in the tumor region of interest was calculated for each photoacoustic image.

### Photoacoustic Tumor Imaging of SWNTs in Living Mice

The synthesis of the QD-RGD probes that were used in the fluorescence tumor targeting experiment is described elsewhere. The mice were inoculated with 10⁷ U87MG cells, and tumors were allowed to grow to 500 mm³. 200 μmol of QD-RGD were injected via the tail vein to the mice. The mice were imaged 6 hr post-injection using the Maestro (CRI) fluorescence imaging instrument. Excitation filter of 575-605 nm, emission long pass filter of 645 nm and liquid crystal filter range between 650 nm to 850 nm were used for this scan.

### Tumour Ex-Vivo Analysis Using Raman Microscopy.

At the conclusion of photoacoustic studies (4 hr post-injection) the mice were sacrificed and the tumors were surgically removed. The tumors were then scanned using a Raman Microscope (Renishaw Inc.). This microscope has a laser operating at 785 nm with a power of 60 mW. A computer-controlled translation stage was used to create a two dimensional map of the SWNT signal in the excised tumors with 750 μm step size using 12× open field lens.

Quantification of the Raman images was performed by using the NANOPLEX™ SENSERSee software (Oxonica Inc.) where the mean Raman signal detected from the tumors was calculated.

We claim the following:

1. A photoacoustic probe comprising:
a carbon nanotube and a plurality of dye molecules bound to said carbon nanotube, wherein the probe has the characteristic of being able to absorb optical energy and to convert the absorbed optical energy to emitted thermal energy.

2. The photoacoustic probe according to claim 1, wherein the emitted thermal energy has the characteristic of being able to generate an acoustic signal in an aqueous medium.

3. The photoacoustic probe according to claim 1, further comprising a targeting moiety bound to the carbon nanotube.

4. The photoacoustic probe according to claim 1, wherein the carbon nanotube is a single-walled nanotube (SWNT).

5. The photoacoustic probe according to claim 1, wherein the carbon nanotube is a multi-walled nanotube (MWNT).

6. The photoacoustic probe according to claim 1, wherein the carbon nanotube has a diameter of about 0.6 nanometers (nm) to about 100 nm, and wherein the carbon nanotube has a length of about 50 nm to about 1 mm.

7. The photoacoustic probe according to claim 1, wherein the carbon nanotube has a diameter of about 2 nanometers (nm) to 5 nm, and wherein the carbon nanotube has a length of about 50 nm to about 500 nm.

8. The photoacoustic probe according to claim 1, wherein the dye compound is selected from the group consisting of: a diarylrhodamine, a polyaromaticazo quencher, Blackberry Q, a bisazulene, an indocyanine, an indocyanine, a dabcyl, a non-fluorescent pocilloporins, an isothiocyanate dye, a multi-sulfur organic dye, a multi-heterosulfur organic dye, a benzotriazole dye, a thiacyanine dye, a dithiacyanine dye, a thiacarbocyanine dye, a dithiacarbocyanine dye, a malachite green isothiocyanate, a tetramethylrhodamine-5-isothiocyante, an X-rhodamine-5-isothiocyanate, an X-rhodamine-6-isothiocyanate, a 3,3′-diethylthiadicarbocyanine iodide, and a combination thereof.

9. The photoacoustic probe according to claim 8, wherein the diarylrhodamine derivatives is selected from the group consisting of: QSY-7, QSY-9, QSY-219.

10. The photoacoustic probe according to claim 8, wherein the polyaromatic-azo quencher is selected from the group consisting of: QSY-35, BHQ-1, BHQ-2 and BHQ-3.

11. The photoacoustic probe according to claim 8, wherein the indocyanine dyes is an indocyanine green dye or a derivative thereof.

12. The photoacoustic probe according to claim 8, wherein the dye molecule is QSY21, and wherein the probe absorbs energy at about 707 nm.

13. The photoacoustic probe according to claim 8, wherein the dye molecule is Indocyanine Green, and wherein the probe absorbs energy at about 780 nm.

14. The photoacoustic probe according to claim 3, wherein the targeting moiety comprises a peptide having the amino acid sequence arginine-glycine-aspartic acid (RGD).

15. The photoacoustic probe according to claim 3, wherein the targeting moiety is selected from the group consisting of: a monoclonal antibody, a polyclonal antibody, an Fab fragment, an Fab′ fragment, an F(ab′)₂ fragment, a single chain Fv (ScFv) fragment, an Fv fragment, a nucleic acid, a polysaccharide, a sugar, a fatty acid, a steroid, a purine, a pyrimidine, and a small molecule ligand.

16. The photoacoustic probe of claim 3, wherein the targeting moiety is bound to the carbon nanotube via a linker.

17. The photoacoustic probe of claim 3, wherein the targeting moiety is bound to the carbon nanotube via a polyethylene glycol (PEG) polymer linker.

18. The photoacoustic probe of claim 1, wherein the photoacoustic probe is in a probe composition, and wherein the probe composition further comprises a pharmaceutically acceptable carrier.

19. A method of detecting a target in a subject, comprising:
delivering a photoacoustic probe to a subject, wherein the photoacoustic probe comprises a carbon nanotube, a plurality of dye molecules, and a targeting moiety, wherein the plurality of dye molecules and the targeting moiety are bound to said carbon nanotube, and wherein the probe has the characteristic of being able to absorb optical energy and being able to convert the absorbed optical energy to emitted thermal energy to produce an acoustic signal in an aqueous medium;
allowing the photoacoustic probe to selectively bind to a target of the subject;
illuminating the system with an optical energy absorbable by the photoacoustic probe, thereby generating an acoustic signal; and
detecting the acoustic signal, thereby detecting the target in the subject.

20. The method of claim 19, wherein detection of the acoustic signal is used to determine the presence and location of the target in the subject.

21. The method of claim 19, further comprising generating an image of the target by detecting the acoustic signal in the subject.

22. A kit comprising a photoacoustic probe according to claim 1, packaging, and instructions for the use of the photoacoustic probe for the enhanced photoacoustic imaging of a region of a subject human or animal.
